(19) United States
(12) Patent Application Publication
(10) Pub. No.: US 2022/0128459 A1
(43) Pub. Date: Apr. 28, 2022

(54) METHOD FOR MEASURING THE CONCENTRATION OF GASEOUS SPECIES IN A BIOGAS

(71) Applicant: IFP Energies nouvelles, RUEIL-MALMAISON CEDEX (FR)

(72) Inventors: Matthieu LECOMPTE, RUEIL-MALMAISON CEDEX (FR); Philipp SCHIFFMANN, RUEIL-MALMAISON CEDEX (FR); Olivier LAGET, RUEIL-MALMAISON CEDEX (FR); Noemie CAILLOL, RUEIL-MALMAISON CEDEX (FR)

(21) Appl. No.: 17/428,238

(22) PCT Filed: Jan. 14, 2020
(86) PCT No.: PCT/EP2020/050834

§ 371 Date: Aug. 3, 2021

(30) Foreign Application Priority Data
Feb. 7, 2019 (FR) 19/01.225

(51) Int. Cl. G01N 21/33; G01N 33/00; G01N 21/85

(52) U.S. Cl. CPC G01N 21/33; G01N 33/0044; G01N 2021/8578; G01N 21/85; G01N 2201/0636; G01N 33/0042

(57) ABSTRACT

The invention relates to a method for in-situ measurement of the concentration of gaseous chemical species contained in a biogas (10) flowing in a pipe (20), for example in a biogas treatment plant or a system using biogas.

The method according to the invention is implemented by means of an optical measurement system (40) including a light source (41) and a spectrometer (44). Source (41) emits a UV radiation (42) through the biogas (10) within a measurement zone (21) in the pipe. Spectrometer (44) detects at least part of said UV radiation that has passed through biogas (10) and it generates a digital signal of the light intensity (50) as a function of the wavelength of the part of the UV radiation that has passed through the biogas. The chemical species concentration is then determined from digital light intensity signal (50).

### FIELD OF THE INVENTION

The present invention relates to the measurement of the concentration of chemical species contained in a biogas, by means of an optical system. The present invention is advantageously applied but not limited to the field of biogas treatment, which aims to convert a biogas to biomethane, and to the use of this biomethane.

Biogas is the product of the anaerobic digestion of waste of organic origin, such as sewage sludge, agricultural waste, landfills. Biogas mainly consists of methane (40% to 70%), CO₂ and water vapour, but it also contains impurities such as sulfur compounds (H₂S, SO₂, . . . ), siloxanes, halogens or VOCs (Volatile Organic Compounds). Therefore, biogas cannot be directly exploited.

In order to be able to exploit biogas, it needs to be cleaned (or purified), notably in order to remove the carbon dioxide and the hydrogen sulfide, as well as the other impurities it contains. Biomethane is thus obtained, which can be injected into the natural gas distribution network or used as biofuel.

A particular use of a purified biogas is the fuel cell, for which the impurity or contaminant tolerance thresholds are particularly high in order not to damage the system (see for example the document “Biogas and fuel cells workshop”, Argonne, 2012, Dennis Papadias and Shabbir Ahmed, Argonne National Laboratory, presented at the Biogas and Fuel Cells Workshop Golden, Colo., Jun. 11-13, 2012).

The development of sensors and methods of measuring each of the polluting substances thus is of major interest in order to control the biogas treatment process and the qualification of the biomethane obtained after purification for the use thereof.

### BACKGROUND OF THE INVENTION

Document DE-2020/08,003,790 U1 is known, which relates to a device and to a method for measuring concentrations of contaminants contained in a biogas. More specifically, a partial biogas stream is permanently passed through a gas cell by means of a pump, then a spectrum, notably an ultraviolet spectrum, is measured by means of a spectrometer. This spectrum is then analysed according to one or more chemometric calibration models. Thus, this method comprises a step of sampling the gas to be analysed. The device described in this document therefore requires extra elements (notably a pump) in addition to the measurement itself, making the device more bulky, expensive, and requiring more maintenance work. Furthermore, analysis of the contaminants is de facto a remote analysis, therefore deferred, which may be damaging notably in the case of a fuel cell. Besides, the method described in this document requires a prior increase in the chemical species concentration to be measured, by means of an adsorption device with a filter, when the species concentrations to be measured are too low to be detected and measured by a spectrometer.

The method according to the invention aims to overcome these drawbacks. Notably, the method according to the invention aims to provide an in-situ optical measurement of the concentration of gaseous chemical species contained in a biogas, without requiring a step of over-concentration of the chemical species present in low amounts in the biogas to be analysed. Furthermore, the method according to the invention enables differentiated and simultaneous measurement of the various gaseous chemical species contained in the biogas. Finally, the method according to the invention can enable diagnosis and/or control of a biogas purification method from measurements of the concentration of the gaseous chemical species contained in the biogas performed before, during and after purification of the biogas. The method according to the invention can also be advantageously implemented upstream from a plant using a biomethane, in a fuel cell for example, so as to guarantee the integrity of the system using this gas.

### SUMMARY OF THE INVENTION

The present invention relates to a method for in-situ measurement of the concentration of at least one gaseous chemical species contained in a biogas flowing in a pipe by means of an optical measurement system comprising at least one light source emitting a UV radiation and at least one spectrometer capable of analysing at least said UV radiation, said pipe comprising at least a first optical access provided in a wall of said pipe.

The method according to the invention comprises at least the following steps:

a) by means of said light source, emitting, at least at said optical access, said UV radiation through said biogas in a measurement zone located at least partly in said pipe,

b) by means of said spectrometer, measuring, at said first optical access and/or at a second optical access, at least part of said UV radiation that has passed through said biogas in said measurement zone, and generating a digital signal of the light intensity as a function of the wavelength (W) of said part of the UV radiation that has passed through said biogas, and

c) determining said concentration of said chemical species contained in said biogas from at least said digital signal.

According to an implementation of the method, step c) can comprise at least:
determining the absorbance of said biogas as a function of the wavelength from said digital signal of the light intensity as a function of the wavelength of said part of the UV radiation that has passed through said biogas and from a digital reference signal of the light intensity as a function of the wavelength predetermined for a reference gas, and
determining said concentration of said at least one chemical species from said absorbance of said biogas, predetermined absorbance characteristics of said chemical species, and an estimation of the temperature and pressure of said biogas.

Advantageously, said absorbance of said biogas can depend on the absorbance length, on the number density of the molecules of said chemical species and on the molar extinction coefficient.

According to an implementation of the invention, said digital reference signal can be obtained by emitting said UV radiation through said reference gas and by measuring at least part of said UV radiation that has passed through said reference gas, said gas having a known or zero concentration in said chemical species.

According to an implementation of the invention, in step c), a temperature of said biogas can further be determined from said digital signal.

According to an implementation of the invention, said temperature can be determined by modification of the molar extinction coefficient of the absorbance of said chemical species extracted from said absorbance of said biogas, said modification being a wavelength offset or a change in amplitude, or a combination of both.

According to an implementation of the invention, said optical measurement system can further comprise a reflector arranged in said measurement zone of said pipe. According to this implementation, it is possible to measure at least at said first optical access at least part of the UV radiation that has been emitted by said light source at the first optical access and that has at least partly reflected on said reflector.

According to an implementation of the invention, said first and/or second optical accesses can be offset with respect to said wall of said pipe in which the biogas flows.

According to an implementation of the invention, said UV radiation can be emitted at a wavelength ranging between 180 and 400 nm, preferably ranging between 180 and 280 nm, and more preferably ranging between 180 and 240 nm.

According to an implementation of the invention, the concentration of at least one and preferably more gaseous chemical species contained in said biogas and included in the list consisting of: SO₂, H₂S, NH₃, BTEX, siloxanes and halogens, can be measured.

According to an implementation of the method, the concentration of at least two gaseous chemical species, preferably at least the H₂S concentration and the NH₃ concentration, can be simultaneously measured.

According to an implementation of the invention, the concentration of at least one gaseous chemical species selected from among the sulfur-containing chemical species SO₂ and H₂S can be measured, and preferably both.

According to an implementation of the invention, the concentration of at least NH₃ can be measured.

According to an implementation of the invention, said pipe in which said biogas flows can be a pipe of a plant for purification of said biogas, and said method can be implemented upstream and/or downstream from said plant.

According to an implementation of the invention, said pipe in which said biogas flows can be a pipe of a system using said biogas, such as a distribution network for said biogas, a vehicle or a fuel cell, and said method can be implemented upstream from said system using said biogas.

### BRIEF DESCRIPTION OF THE FIGURES

Other features and advantages of the invention will be clear from reading the description hereafter of particular embodiments of the invention, given by way of non-limitative example, with reference to the accompanying figures wherein:

FIG. 1A is a diagram illustrating the optical measurement of the concentration of chemical species contained in a biogas, according to a transmissive configuration of the optical measurement system for implementing the method according to the invention,

FIG. 1B is a diagram illustrating the optical measurement of the concentration of chemical species contained in a biogas, according to a reflective configuration of the optical measurement system for implementing the method according to the invention,

FIGS. 1C and 1D respectively show variants of the embodiments illustrated in FIGS. 1A and 1B, comprising optical accesses offset with respect to the pipe in which the biogas flows,

FIG. 2 schematically shows the absorbance of the biogas comprising various gaseous chemical species A, B, C to be measured,

FIG. 3 schematically shows the influence of temperature on the absorbance of a given chemical species contained in the biogas, and

FIGS. 4 to 14 are diagrams illustrating various embodiments of the optical measurement method for implementing the method according to the invention.

### DETAILED DESCRIPTION OF THE INVENTION

The present invention relates to a method for in-situ measurement of the concentration of at least one gaseous chemical species contained in a biogas by means of an optical measurement system.

Biogas is understood to be any gas resulting from the anaerobic digestion of waste of organic origin, such as sewage sludge, agricultural waste, landfills. Biomethane therefore is a biogas according to the invention.

The present invention enables in-situ measurement, i.e. directly in a pipe in which the biogas flows and without taking a biogas sample(s). This pipe can be a pipe in a plant for treating said biogas (a pipe of a biogas purification plant for example) and/or a pipe located upstream from a plant using a biogas (a line of a biogas distribution network for example). More generally, it is referred to hereafter as the pipe of a plant to be monitored.

Furthermore, as described below, the present invention requires no biogas preconditioning (by over-concentration for example) in the case of a chemical species present in low amounts in the biogas to be analysed.

The method according to the invention is implemented by means of an optical measurement system comprising at least a light source emitting a UV radiation and a spectrometer. According to the invention, the pipe in which the biogas flows comprises at least one optical access provided in the pipe in which the biogas flows, said optical access being capable of allowing at least UV rays to pass through. This optical access can consist of an opening provided in the pipe, on which a lens or a porthole is fastened for example.

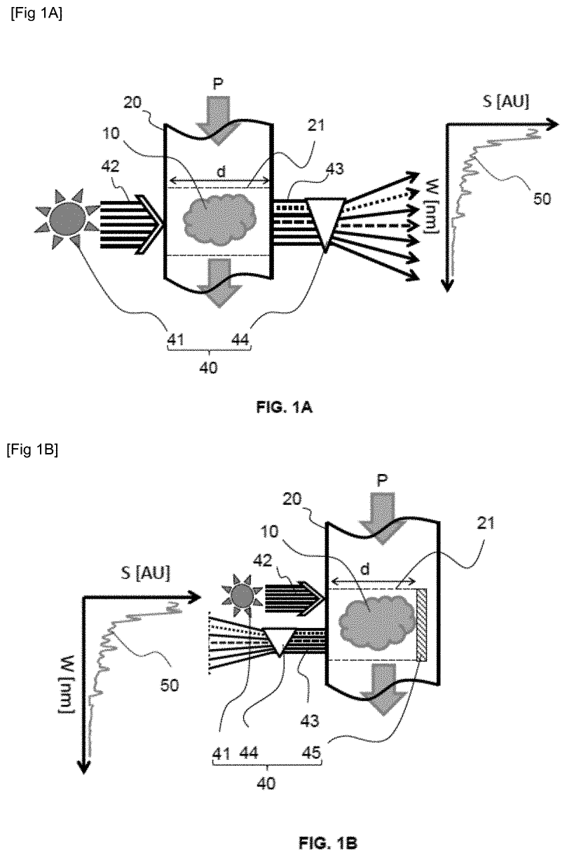

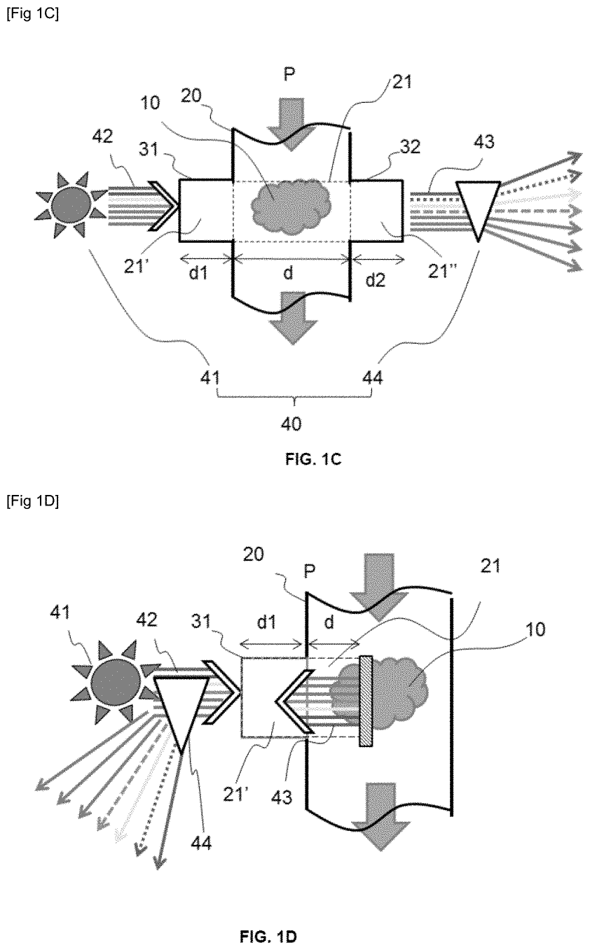

FIGS. 1A and 1B schematically show, by way of non-limitative example, the measurement principle according to the invention. FIG. 1A differs from FIG. 1B by the optical measurement system, which is shown in a transmissive configuration in FIGS. 1A and in a reflective configuration in FIG. 1B.

The measurement method comprises the following steps:
emitting with a light source 41 a UV radiation 42 through a biogas 10 within a measurement zone 21 located in a pipe 20 (a pipe in a biogas treatment plant for example) in which the biogas flows. UV radiation 42 passes through the biogas, along an optical path of length d, which may be substantially but not limitatively perpendicular to path P of the biogas, as shown in FIGS. 1A and 1B. UV radiation 42 passes into the measurement zone through an optical access, a porthole or a lens for example, provided in the pipe in which the biogas flows,
detecting with spectrometer 44 at least one part 43 of the UV radiation that has passed through the biogas in measurement zone 21, and generating a digital signal 50 of the light intensity as a function of the wavelength of the part of the UV radiation that has passed through the biogas. The gaseous chemical species whose concentration is to be measured absorb part of the UV radiation and each gaseous chemical species absorbs the rays at some given wavelengths. The absorption obeys, under ideal conditions, the Beer-Lambert law. The UV radiation that has passed through the biogas is detected by spectrometer 44 through an optical access (another optical access for the embodiment of FIG. 1A, the same optical access for the embodiment of FIG. 1B), provided as for the emission by the light source,
estimating the concentration [X] of the gaseous chemical species from at least digital signal 50.

While the configuration is transmissive in the embodiment illustrated in FIG. 1A, the configuration is reflective in the embodiment illustrated in FIG. 1B. According to this reflective configuration, optical system 40 further comprises a reflector 45. The UV radiation emitted by source 41 is reflected by reflector 45, positioned at the end of measurement zone 21 opposite to the end comprising light source 41 and spectrometer 44. Reflector 45 is preferably positioned in pipe 20 in which the biogas flows, as shown in FIG. 1B. Alternatively, it may be integrated in the wall of this element, or arranged externally thereof. UV radiation 42 passes a first time through biogas 10 in measurement zone 21, it is reflected by reflector 45, passes a second time through the biogas, in the opposite direction, in measurement zone 21, and it is subsequently detected by spectrometer 44, as described above. Furthermore, the location of reflector 45 can be adjusted depending on the order of magnitude of the chemical species concentrations sought. Indeed, the longer the optical path travelled by the UV radiation through the biogas, the more reliable and accurate the concentration measurement in case of low chemical species concentrations. Mirror 45 can thus be advantageously arranged on the wall of pipe 20 opposite to light source 41, so as to increase (here, to double) the optical path length in relation to the configuration of FIG. 1A.

According to another variant embodiment of the invention, the length of the optical path travelled by the UV radiation in the biogas can be adjusted by means of at least one optical access offset with respect to the wall of the pipe in which the biogas flows. This offset optical access can be a tube fastened at one end thereof to the opening provided in the pipe of the plant to be monitored, and the other end of this tube comprises a means capable of allowing the UV radiation to pass through, such as a porthole or a lens. The biogas flowing in the pipe of the plant to be monitored can therefore also occupy the space defined by said offset optical access fastened to said element, thus enlarging the measurement zone. According to this variant embodiment of the invention, the cross section (relative to the principal direction of the UV radiation) of the offset optical access is preferably substantially circular, but it may have any shape, preferably in accordance with the shape of the opening provided in the pipe of the plant to be monitored. FIG. 1C shows an example embodiment of this variant of the invention, in the case of a transmissive configuration of the optical system according to the invention as defined above, comprising two offset optical accesses 31′, 31″ in form of circular tubes of length d1 and d2 respectively, the first offset optical access being intended for passage of the UV radiation emitted by light source 41 and the second offset optical access being intended for passage of the UV radiation that has passed through the biogas in measurement zone 21, 21′, 21″, for measurement by spectrometer 44. In this case, the total optical path of the UV radiation that has passed through the biogas is d+d1+d2. FIG. 1D shows another example embodiment of this variant of the invention in the case of a reflective configuration of the optical system according to the invention as defined above, comprising an offset optical access 31 in form of a circular tube of length d1 in the longitudinal direction, this optical access being intended for passage of the UV radiation emitted by light source 41, then reflected by reflector 45 after passing a first time through the biogas in measurement zone 21′, 21, and passing again through the optical access to be detected by spectrometer 44 after passing a second time through the biogas in measurement zone 21′, 21. In this case, the total optical path of the UV radiation that has passed through the biogas is 2(d+d1). These various non-limitative configurations of the optical system according to the invention, comprising at least one offset optical access, allow to vary the length of the optical path travelled by the UV radiation and thus to improve the concentration measurement accuracy in case of low chemical species concentrations.

Thus, the method according to the invention, which can be implemented in situ, has the advantage of not modifying the biogas flow and of being instantaneous, for example with a response time that can be less than 0.1 s, unlike known methods using gas sampling, with the addition of back pressure, a possible evolution of the gases to be analysed during sampling, which is unwanted (indeed, during sampling, the gas may condense, which may contribute to modifying the gas that is eventually analysed, for example by adsorption of some molecules on the sample tube walls), and transit of the gases to the measurement cell, causing delayed measurement.

Furthermore, the method according to the invention can enable reliable and accurate measurement of the chemical species present in the biogas in low amounts, without requiring a prior step of over-concentration of the chemical species, by adjusting the length of the optical path depending on the arrangement of the elements of the optical system according to the invention.

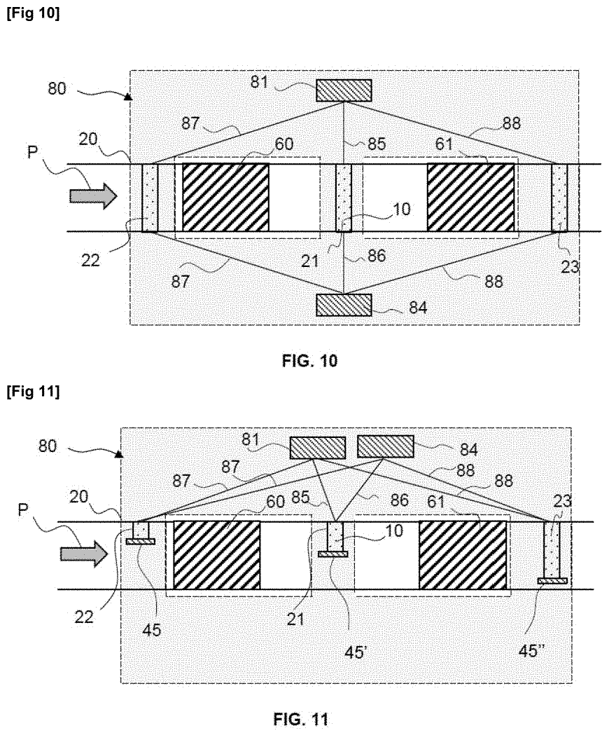

Whatever the configuration, transmissive or reflective, light source 41 and spectrometer 44 are preferably positioned outside pipe 20 in which the biogas flows, for example on the outer face of the pipe walls, or at a distance from this element if radiation transmission means are provided, such as optical fibres for example, as shown in FIGS. 10 and 11 described hereafter. This notably makes it possible to avoid fouling of these optical elements.

The method according to the invention preferably comprises a prior step of calibrating the optical measurement system allowing to obtain a digital reference signal of the light intensity as a function of the wavelength.

Preferably, this step consists in emitting the UV radiation through a reference gas, for example a gas containing none of the chemical species to be measured (such as helium, dinitrogen or air), or through a reference gas containing some chemical species to be measured, whose concentration in said gas is known. The radiation passes through the reference gas and it is subsequently detected by the spectrometer in order to provide a digital reference signal of the light intensity as a function of the wavelength of the part of the UV radiation that has passed through the reference gas. The reference signal is used in the concentration and temperature estimation step, in particular to calculate the biogas absorbance, as described in detail below.

The wavelength of the UV radiation emitted by light source 42 ranges between 180 and 400 nm, preferably between 180 and 280 nm (notably in cases where the chemical species is NO), or more preferably between 180 and 240 nm (notably in cases where the chemical species is NH₃). These wavelength ranges belong to what is known as deep UV.

By way of example, the light source may be a UV light-emitting diode (LED), in particular a deep UV light-emitting diode as mentioned above, or maybe a xenon, deuterium, zinc, cadmium lamp, or another gas lamp such as KrBr, KrCl, KrF excimer lamps.

The spectrometer allows to analyse the light signal in the 180-400 nm wavelength range, preferably in the 180-280 nm range and more preferably in the 180-240 nm range. Alternatively, a simplified system allowing a reduced wavelength range to be analysed can also be used. The term “spectrometer” is kept in the present invention to designate such a simplified system.

The assembly made up of at least the UV light source and the spectrometer, also referred to as optical system or optical sensor in the present invention, is known per se. Such optical sensors can be commercially available.

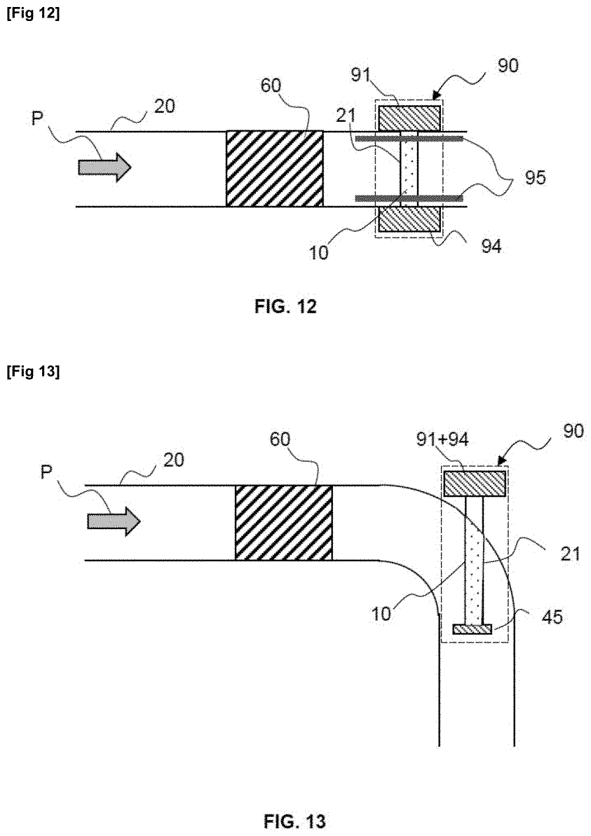

The optical system can comprise other elements, notably optical elements such as lenses for modifying the light beam if need be (convergence or divergence for example), or protective elements intended to protect the light source and the spectrometer, in particular during cold operation of the optical measurement system. Indeed, cold operation can generate deposits on the optical elements due to a condensation phenomenon. Such protective elements are described below in connection with FIG. 12. The position of the sensor provided on the pipe in which the biogas flows can be selected so as to limit fouling thereof.

According to the invention, at least one gaseous chemical species X can be measured, and preferably more gaseous chemical species X from the list consisting of: SO₂, H₂S, NH₃, BTEX (which includes benzene, toluene, ethylbenzene and xylene), siloxanes and halogens. Preferably, at least one, and more preferably more gaseous chemical species from the list as follows are measured: hydrocarbons (such as aromatic compounds, alkenes, terpenes and terpenoids), siloxanes (such as D2 to D7), sulfur-containing organic compounds (such as sulfides, mercaptans, thiols) or inorganic compounds (such as sulfides), halogens. Advantageously, at least the THT (tetrahydrothiophene) concentration is measured.

Advantageously, differentiated and simultaneous measurement of the concentration of a plurality of these gaseous chemical species can be performed.

Differentiated measurement is understood to provide access to the specific concentration of each chemical species, as opposed to a global measurement of the concentration of several chemical species without distinction. For example, the concentration of at least two gaseous chemical species is simultaneously measured according to the invention, preferably at least the H₂S concentration and the NH₃ concentration.

According to an implementation of the invention, the concentration of at least SO₂ or H₂S, and preferably at least both, can also be measured. Quantification of the sulfur elements in a biogas is particularly useful when the method according to the invention is implemented to qualify the biogas before using it in a fuel cell, for which corrosion may be very harmful.

Advantageously, the concentration of at least NH₃ is measured. By repeating the steps of the method according to the invention at different times, it is for example possible to monitor the evolution over time of the NH₃ concentration of a biogas purification plant.

In the method according to the invention, the concentration of each chemical species is determined from the optical measurement performed on the biogas and from an optical signature specific to each chemical species. Each gaseous chemical species whose concentration is to be measured indeed absorbs part of the UV radiation and therefore has an absorption spectrum of its own (absorbance as a function of wavelength).

During the step of estimating the concentration [X] of at least one chemical species, steps a) and b) described below are carried out:

a) determining the absorbance A of the biogas as a function of wavelength W, from the digital light intensity signal 50 generated by the spectrometer and resulting from the detection of part of the UV radiation that has passed through the biogas, and from a digital reference signal. The digital reference signal is preferably established during the prior calibration step described above. In particular, the biogas absorbance is calculated with a formula of the type of formula (I) hereafter:

$$Absorbance = - ln ( \frac{signal \text{?}}{{signal}_{reference}} ) \\ \text{?} \text{indicates text missing or illegible when filed} \qquad ( I )$$

b) determining, by means of signal analysis and processing means such as a microprocessor, the concentration [X] of each chemical species to be measured, from biogas absorbance A, from predetermined absorbance characteristics and from an estimation of the pressure and temperature of each chemical species. These predetermined absorbance characteristics of each chemical species are preferably obtained during prior measurement campaigns allowing a library to be created. Data from the literature can also be supplied to such a library. The absorbance characteristic of a given chemical species is understood to be the molar extinction coefficient thereof. Advantageously, the pressure and/or the temperature can be estimated by measurement during the implementation of the method according to the invention, using a pressure sensor and/or a temperature sensor respectively. Advantageously, according to an implementation of the invention, the temperature of the biogas is estimated by means of the main variant described hereafter, which may be an additional step c) in relation to steps a) and b) described above.

According to a main variant of the method according to the invention, the temperature (T) of the biogas flowing in the pipe is further determined in addition to the concentration. According to an implementation of this main variant, temperature (T) of the biogas flowing in the pipe is determined by modification of the molar extinction coefficient of the absorbance of the chemical species whose concentration is to be measured, said absorbance of the chemical species being extracted from the absorbance of said biogas. The molar extinction coefficient modification can be a wavelength offset, leading to an absorption at different wavelengths, or a change in amplitude of the absorbance at a given wavelength, or a combination thereof. When the exact behaviour of the molar extinction coefficient of the absorbance as a function of the temperature of a chemical species is known, through the agency of prior measurements or of data from the literature, allowing a library to be created, this chemical species can be used as a temperature indicator. The degree of accuracy in the determination of the temperature depends on the sensitivity of the molar extinction coefficient of the chemical species in the measured wavelength range. FIG. 3 illustrates the influence of temperature on the absorbance of a chemical species, ammonia here, used to determine the temperature according to the present invention. Curve A-Tc shows the absorbance of NH₃ for a low temperature, 20° C. for example, and curve A-Th shows the absorbance of NH₃ for a high temperature, 450° C. for example. A modification of the molar extinction coefficient leads for example to an offset of the absorption signal. Although this example uses ammonia, any other chemical species such as SO₂, H₂S, NH₃, BTEX, siloxanes, halogens, aldehydes such as acetaldehyde or formaldehyde, non-aromatic hydrocarbons such as acetylene or buta-1,3-diene can be used to determine the temperature. The same type of algorithms as those used to determine the concentration of the chemical species can be used to determine the temperature.

Thus, the method according to this main variant of the invention makes it possible to access the temperature of the biogas without any additional measuring device in the control zone. Furthermore, this main variant enables instantaneous temperature measurement by means of a specific UV absorption signal processing, simultaneously with the measurement of the concentration of gaseous chemical species contained in the gases.

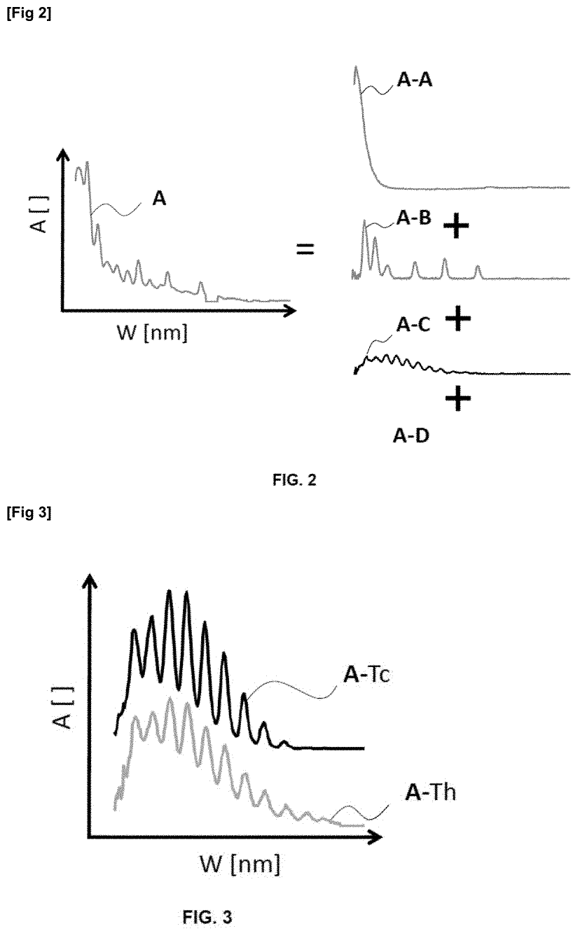

FIG. 2 schematically shows absorbance A of the biogas comprising various gaseous chemical species A, B, C to be measured. The diagram on the left shows an example of absorbance A (unitless) of the biogas, expressed as a function of wavelength W (in nm), calculated from the digital light intensity signal 50 generated by the spectrometer and from the digital reference signal.

The absorbance A of a gas depends on the absorbance length, i.e. the length of the optical path travelled by the light in the measurement zone, on the number density of the molecules of the gaseous chemical species (A, B, C) contained in the gas and on the molar extinction coefficient of the chemical species. The molar extinction coefficient, also referred to as molar absorptivity, is a measurement of the probability that a photon interacts with an atom or a molecule.

The number density of the molecules of a chemical species itself depends on the temperature, the pressure and the concentration of the chemical species, and the molar extinction coefficient depends on the wavelength, on the chemical species, on the temperature and on the pressure.

Thus, knowing the predetermined molar extinction coefficient, temperature and pressure characteristics of each chemical species makes it possible to determine the concentration [X] of each chemical species X from absorbance A of the biogas. The absorbance values of each chemical species add up and their sum is substantially equal (apart from the noise) to the absorbance values A of the biogas. This is shown on the right side of FIG. 2 by absorbance diagrams A-A, A-B and A-C of the chemical species A, B and C to be detected, which add to the noise and to the absorbance of the undetected species A-D to form absorbance A of the biogas.

Various types of algorithm can be used to determine the concentration values, such as least squares adjustment algorithms applied to the absorbance signals themselves, to the derivatives of the absorbance signals or to the frequency portion of the absorbance signals (typically derived from a Fourier transform). Similarly, a certain number of chemometric methods can be used for this process such as, for example, principal component analysis (PCA) or partial least squares (PLS) algorithms.

The invention advantageously applies to the field of biogas treatment, which can comprise a biogas purification (or cleaning) plant. Within this context, the optical measurement system according to the invention can be positioned in different places of a biogas purification plant, notably upstream and/or downstream from such a plant. This makes it possible to control the quality of a biogas treating method, at different stages of this treatment and, moreover, in real time. The method according to the invention can also be advantageously applied upstream from a system using a biogas, notably an already purified biogas, so as to ensure that the biogas used in said system complies with the operating and/or regulatory requirements of this system.

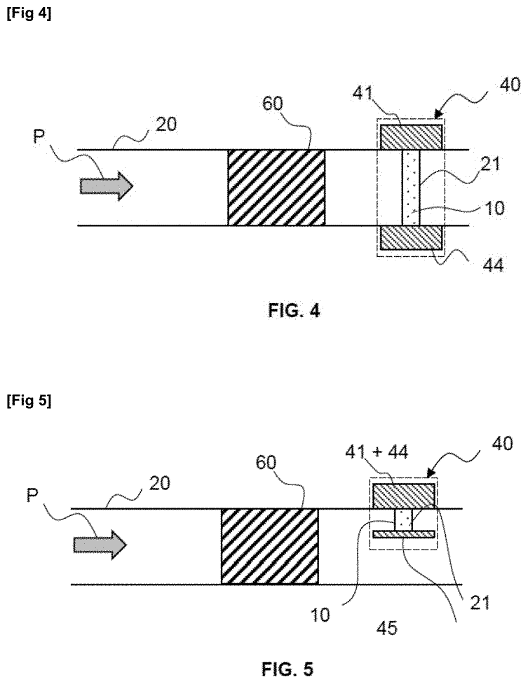

According to one embodiment, the measurement is performed downstream from a biogas purification plant and/or a system using a biogas. Such an embodiment is schematically illustrated in FIG. 4. Biogas 10 flows along path P in a pipe 20 of a biogas purification plant 60. According to this embodiment, optical measurement system 40 is arranged downstream from biogas purification plant 60. The upstream and downstream positions are defined in relation to the direction of flow of the biogas in pipe 20. Measurement of the concentration of some chemical species of the biogas downstream from a biogas purification plant 60 allows to check whether the pollutant emission standards are met by such plants, to monitor their evolution and, if necessary, to adjust the operation of these plants so as to comply with the standards in force.

According to another embodiment, shown in FIG. 5, the in-situ measurement is performed similar to that of the embodiment illustrated in FIG. 4, except that the optical measurement system comprises a reflector 45 for reflective measurement. The optical measurement system is the one described in connection with FIG. 2. Source 41 and spectrometer 44 are arranged on the same side of pipe 20, i.e. at the same end of measurement zone 21 opposite to the end where reflector 45 is positioned.

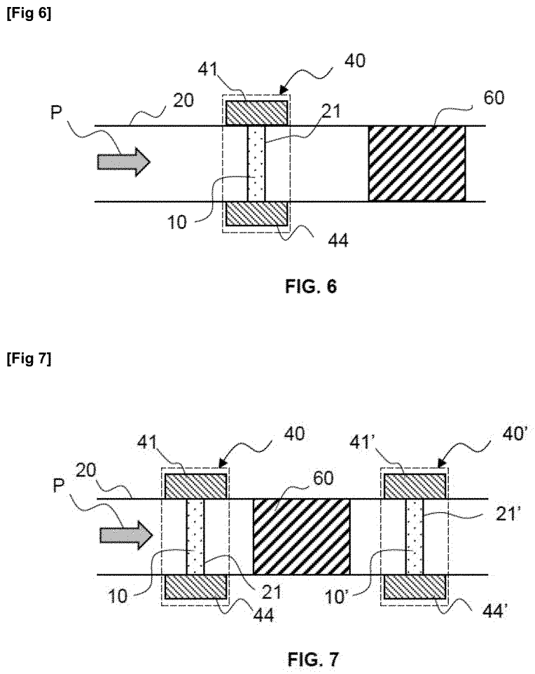

According to another embodiment, the in-situ measurement is performed upstream from at least one plant to be monitored, such as a biogas purification plant or a system using a biogas. Such an embodiment is schematically shown in FIG. 6, identical to FIG. 4 except for optical measurement system 40 positioned upstream from plant 60 to be monitored. Such an embodiment can be useful to obtain information on the concentration of chemical species in the biogas before they are fed to a biogas purification plant, so as to influence the operation of this plant for example. Such an embodiment can also be advantageously implemented upstream from a system using a biogas, such as a distribution network for said biogas, a vehicle or a fuel cell, in order to control in real time the quality of the biogas entering this system.

According to one embodiment, the in-situ measurement is performed both upstream and downstream from at least one plant to be monitored, such as a biogas purification plant. An example according to this embodiment is illustrated in FIG. 7, where two optical measurement systems 40 and 40′ are respectively arranged upstream and downstream from a purification plant 60. The second optical measurement system 40′ is identical to first optical measurement system 40 arranged upstream, and it comprises a light source 41′ and a light analyzer 44′ respectively providing emission of the UV radiation and detection and analysis of the UV radiation that has passed through the biogas in measurement zone 21′ located along pipe 20, so as to provide an estimation of the concentration of gaseous chemical species.

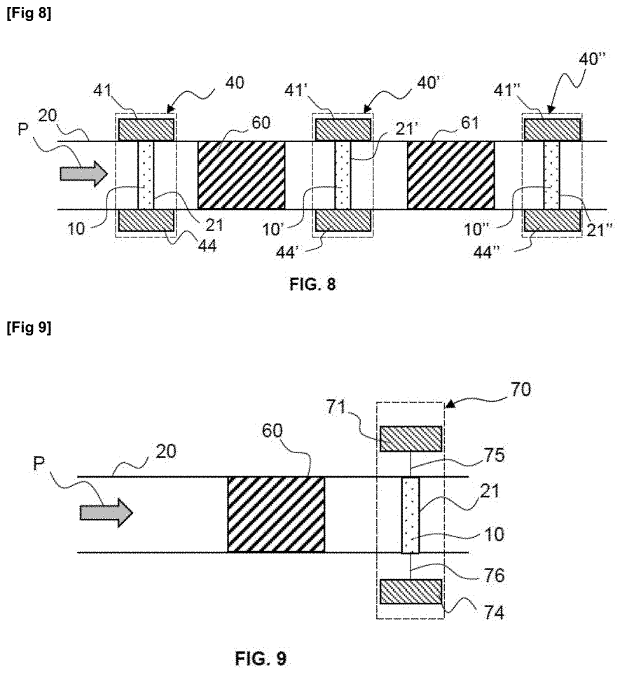

Another example according to this embodiment is illustrated in FIG. 8, where the method according to the invention is implemented by means of three optical measurement systems 40, 40′ and 40″, respectively arranged upstream from a first biogas purification plant 60, between first purification plant 60 and a second purification plant 61, and downstream from second biogas purification plant 61. Advantageously, first plant 60 is a plant using a first type of biogas treatment, and second plant 61 is a plant using a second type of biogas treatment. Such an embodiment allows to monitor the efficiency of each plant for converting the biogas to biomethane, and possibly to adjust the parameters of these plants according to the concentration information obtained upstream from, downstream from and between the various plants.

According to one embodiment, the UV light source and/or the spectrometer of the optical measurement system is connected to the pipe in which the biogas flows by an optical fibre, allowing for example the optical measurement elements to be arranged in a protective enclosure without affecting the instantaneous measurements. An example of such an embodiment is shown in FIG. 9, where optical fibres 75 and 76 respectively connect light source 71 and spectrometer 74 of optical measurement system 70 to pipe 20, at measurement zone 21 where the biogas is traversed by the UV radiation conveyed by optical fibres 75 and 76. It is clear that optical fibre 76 connected to spectrometer 74 can be (not shown) arranged on the same side of pipe as optical fibre 75 connected to light source 71.

It is clear that, for each one of the embodiments shown in FIGS. 6 to 9, the optical system can be the one described in connection with FIG. 1B, comprising at least one reflector located in the measurement zone. In the case of the embodiments shown in FIGS. 6 to 8, spectrometer(s) 44, 44′, 44″ can then be arranged (not shown) on the same side of pipe 20 as light source(s) 41, 41′ and 41″.

According to one embodiment, the optical measurement system comprises several measurement zones connected to a single light source and a single spectrometer, by optical fibres. Such an embodiment allows for example to reduce the cost of implementing the optical measurement in cases where measurements upstream and downstream from a plant to be monitored (a purification plant for example) are desired. An example of such an embodiment is illustrated in FIG. 10, where optical measurement system 80 comprises three measurement zones 21, 22 and 23, a single light source 81 and a single spectrometer 84 connected to the measurement zones by optical fibres 85, 86, 87 and 88.

According to another embodiment, the in-situ measurement is performed similar to that of the embodiment illustrated in FIG. 10, except that the optical measurement system comprises three reflectors 45, 45′ and 45″ as described in connection with FIG. 2, respectively arranged at the ends of measurement zones 21, 22 and 23. As schematically illustrated in FIG. 11, the length of measurement zones 21, 22 and 23 increases in the upstream-downstream direction; in other words, the length of the optical path increases in the upstream-downstream direction. This embodiment is particularly advantageous for improving the accuracy of measurement of the efficiency of a biogas treatment method because, as the biogas flows through biogas purification plants (plants 60 and 61 here), the chemical species are present in the biogas at increasingly lower concentrations. Therefore, it may be advantageous to adjust the optical path of the UV radiation, notably by lengthening it in the downstream direction, so as to enable reliable measurement of the chemical species concentration, even in the case of a low chemical species concentration. This avoids the use of devices for over-concentrating the chemical species, notably at the end of a biogas treatment process. In order to lengthen the optical path in the upstream-downstream direction, it is possible, alternatively or in combination with the embodiment of FIG. 10, to implement the method according to one of the embodiments of the invention described above for which the optical system comprises at least one offset optical access.

According to an embodiment illustrated in FIG. 12, optical measurement system 90 comprises means 95 for protecting light source 91 and/or spectrometer 94. Such protective means are useful for example during cold operation of the optical measurement system, to prevent fouling of the optical elements, as already explained above. These protective means can include a flap, an air barrier between light source 91 or spectrometer 94 and the biogas flowing through measurement zone 21, a specific coating, to prevent liquid or solid particles adhesion for example, on the surface(s) between light source 91 and the biogas or between spectrometer 94 and the biogas, or a means of heating said surfaces. It may also be a specific geometry of the optical sensor, not shown in FIG. 12.

According to one embodiment, illustrated in FIG. 13, the configuration of optical measurement system 90 is of reflective type, optical measurement system 90 comprising a light source 91, a spectrometer 94 and a reflector 45, these elements of optical measurement system 90 being positioned for example at a bend of pipe 20, so that the optical path of the UV radiation is substantially tangent to path P of the biogas in measurement zone 21.

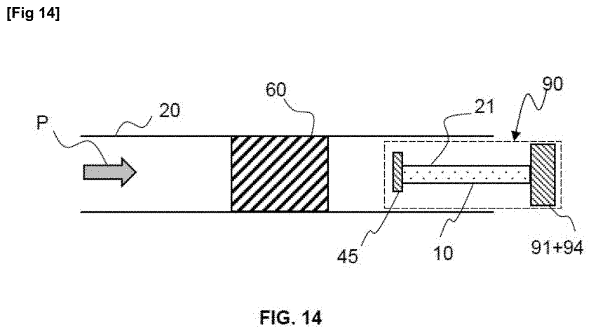

According to another embodiment, illustrated in FIG. 14, the configuration of optical measurement system 90 is of reflective type, optical measurement system 90 comprising a light source 91, a spectrometer 94 and a reflector 45, these elements of optical measurement system 90 being arranged within pipe 20, preferably at the outlet of pipe 20, so that the optical path of the UV radiation is substantially parallel to path P of the biogas in measurement zone 21.

The invention further relates to a plant comprising at least one optical measurement system as described above for implementing the method according to the invention. According to one implementation of the invention, the plant can be a biogas purification plant and/or a system using a biogas. According to an implementation of the invention wherein the plant is a system using a biogas, the method according to the invention can be implemented upstream from said plant.

The invention further relates to a use of the method according to the invention for measuring the concentration of at least one gaseous chemical species contained in a biogas flowing in a pipe of a plant. According to one implementation of the invention, the plant can be a biogas purification plant and/or a plant using a biogas. According to an implementation of the invention wherein the plant is a system using a biogas, the method according to the invention can be implemented upstream from said plant.

1. A method for in-situ measurement of the concentration ([X]) of at least one gaseous chemical species contained in a biogas flowing in a pipe by means of an optical measurement system, comprising at least one light source emitting a UV radiation and at least one spectrometer capable of analysing at least the UV radiation, the pipe comprising at least a first optical access provided in a wall of the pipe, the method comprising at least the following steps:
a) by means of the light source, emitting, at least at the optical access, the UV radiation through the biogas in a measurement zone located at least partly in pipe,
b) by means of the spectrometer, measuring, at the first optical access and/or at a second optical access, at least part of the UV radiation that has passed through the biogas in the measurement zone, and generating a digital signal of the light intensity as a function of the wavelength (W) of the part of the UV radiation that has passed through the biogas, and
c) determining the concentration ([X]) of the chemical species contained in the biogas from at least the digital signal.

2. A method as claimed in claim 1, wherein step c) comprises at least:
determining the absorbance of the biogas as a function of the wavelength from the digital signal of the light intensity as a function of the wavelength of the part of the UV radiation that has passed through the biogas and from a digital reference signal of the light intensity as a function of the wavelength predetermined for a reference gas, and
determining the concentration ([X]) of the at least one chemical species from the absorbance of biogas, predetermined absorbance characteristics of the chemical species, and an estimation of the temperature and pressure of the biogas.

3. A method as claimed in claim 2, wherein the absorbance (A) of the biogas depends on the absorbance length, on the number density of the molecules of the chemical species and on the molar extinction coefficient.

4. A method as claimed in claim 2, wherein the digital reference signal is obtained by emitting the UV radiation through the reference gas and by measuring at least part of the UV radiation that has passed through the reference gas, the gas having a known or zero concentration in the chemical species.

5. A method as claimed in claim 1 wherein, in step c), a temperature (T) of the biogas is further determined from the digital signal.

6. A method as claimed in claim 5, wherein the temperature (T) is determined by modification of the molar extinction coefficient of the absorbance of the chemical species extracted from the absorbance of the biogas, the modification being a wavelength offset or a change in amplitude, or a combination of both.

7. A method as claimed in claim 1, wherein the optical measurement system further comprises a reflector arranged in the measurement zone of the pipe and wherein, in step b), it is possible to measure at least at the first optical access at least part of the UV radiation that has been emitted by the light source at the first optical access and that has at least partly reflected the reflector.

8. A method as claimed in claim 1, wherein first and/or second optical accesses are offset with respect to the wall of the pipe in which biogas flows.

9. A method as claimed in claim 1, wherein the UV radiation is emitted at a wavelength ranging between 180 and 400 nm, preferably ranging between 180 and 280 nm, and more preferably ranging between 180 and 240 nm.

10. A method as claimed in claim 1, wherein the concentration ([X]) of one or more gaseous chemical species SO₂, H₂S, NH₃, BTEX, siloxanes, and halogens contained in the biogas is measured.

11. A method as claimed in claim 1, wherein the concentration ([X]) of at least two gaseous chemical species, preferably at least the H₂S concentration and the NH₃ concentration, is simultaneously measured.

12. A method as claimed in claim 1, wherein the concentration of at least one gaseous chemical species selected from among the sulfur-containing chemical species SO₂ and H₂S, and preferably both, is measured.

13. A method as claimed in claim 1, wherein the concentration of at least NH₃ is measured.

14. A method as claimed in claim 1, wherein the pipe in which the biogas flows is a pipe of a plant for purification of the biogas, the method is implemented upstream and/or downstream from the plant.

15. A method as claimed in claim 1, wherein the pipe in which the biogas flows is a pipe of a system using the biogas, such as a distribution network for the biogas, a vehicle or a fuel cell, and the method is implemented upstream from the system using the biogas.
